(19) United States
(12) Patent Application Publication
(10) Pub. No.: US 2018/0251396 A1
(43) Pub. Date: Sep. 6, 2018

(54) PROCESS FOR PRODUCING GRANULAR PELLETIZED GLASS MATERIAL WITH TRACE ELEMENTS, ESPECIALLY AS GROWTH SUPPORT FOR SELECTIVE NUTRIENT SUPPLY OF MICROORGANISMS AND GRANULAR PELLETIZED GLASS MATERIAL

(71) Applicant: Dennert Poraver GmbH, Postbauer-Heng (DE)

(72) Inventor: KARL WEINBERGER, BISCHOFSMAIS (DE)

(21) Appl. No.: 15/972,271

(22) Filed: May 7, 2018

Related U.S. Application Data
(63) Continuation of application No. PCT/EP2016/076557, filed on Nov. 3, 2016.

(30) Foreign Application Priority Data
Nov. 5, 2015 (DE) 10 2015 221 778.7

(51) Int. Cl. C03C 11/00; C03C 4/00; C03C 3/087; C03C 1/02; C12M 1/107; C12M 1/12

(52) U.S. Cl. CPC C03C 11/007; C03C 4/0007; C03C 3/087; C03C 1/02; C03C 2203/10; C12M 25/16; C03C 2201/11; C03C 2201/40; C03C 2201/58; C12M 21/04

(57) ABSTRACT

A pelletized expanded glass material is provided, which is particularly suitable as a growth support for microorganisms, especially for use in a biogas plant or an anaerobic sewage treatment plant. The production process of the invention for the pelletized expanded glass material contains the steps of: mixing a ground glass, an expanding agent and a binder to give a starting mixture. The starting mixture is pelletized to give ground glass pellet green bodies. The ground glass pellet green bodies are foamed to give expanded glass pellet particles at temperatures of 600 to 950° C. Accordingly, especially in the production of the starting mixture, minerals and or trace elements are added, which serve especially for the nutrient supply of microorganisms used in the biogas plant or the anaerobic sewage treatment plant.

### CROSS-REFERENCE TO RELATED APPLICATION

This is a continuation application, under 35 U.S.C. § 120, of copending international application No. PCT/EP2016/076557, filed Nov. 3, 2016, which designated the United States; this application also claims the priority, under 35 U.S.C. § 119, of German patent application No. DE 10 2015 221 778.7, filed Nov. 5, 2015; the prior applications are herewith incorporated by reference in their entirety.

### BACKGROUND OF THE INVENTION

### Field of the Invention

The invention relates to a process for producing granular expanded-glass material, especially for use in a biogas plant or an anaerobic wastewater treatment plant. The invention further relates to a granular expanded-glass material especially for use in a biogas plant or an anaerobic wastewater treatment plant.

The current system of energy production is based on the use of fossil fuel. The energy sources used are in some cases exposed to sharp price rises, which result from a forseeable shortage of the resources. Moreover, since fossil energy sources are mainly responsible for the anthropogenic greenhouse effect, there have been increased efforts in recent years to reduce their share of final energy consumption.

At the European level, an agreement was reached in December 2008 on an “integral climate-protection strategy”. The aim is to achieve a reduction in the emission of CO₂ by at least 20%, based on the 1990 level, by 2020 [KIENBERGER Th: 2010].

One of the most promising approaches is the utilization of biomass and the production of biogas that is possible therefrom. They are considered as “CO₂ neutral”, since just as much CO₂ arises during their energetic/thermal utilization as was absorbed beforehand during growth. As a result, a climate-neutral utilization can be ideally achieved.

In Germany at the start of 2012, there were approximately 6900 plants in which biogas is obtained by fermentation of biomass. The conversion of biomass into biogas achieves the highest energy yield per hectare of cultivation area.

In comparison with the second-generation BtL fuels, which will only be commercially available in a few years time, biogas, or biomethane produced therefrom through processing, can already be used today in all natural-gas vehicles without any technical modifications. In appropriate motors, biogas or biomethane can be converted into electricity with very high efficiencies. The range of a biogas-driven passenger vehicle is, in this connection, about 67 600 km per hectare of cultivation area.

In a biogas plant, there is fundamentally the problem that those microorganisms which metabolize biomass to form biogas typically exhibit very low growth rates, and this is associated with a low biomass-to-biogas conversion rate. In order, nevertheless, to ensure an acceptable conversion rate in a biogas plant through a comparatively high concentration of microorganisms, use is frequently made in biogas plants of growth supports, on which the microorganisms settle as biofilm and are thus immobilized. To support the immobilized microorganisms in the reaction space of the biogas plant, a magnetic granular expanded-glass material is, for example, used as growth support. Such a magnetic expanded glass, developed by the applicant, is known from German patent DE 10 2010 039 232 B4 from the applicant.

### SUMMARY OF THE INVENTION

It is an object of the invention to provide a granular expanded-glass material which is particularly suitable as growth support for microorganisms, especially for use in a biogas plant or an anaerobic wastewater treatment plant.

With regard to a process for producing the granular expanded-glass material, the object is achieved according to the invention by the features of the first independent claim. With regard to the granular expanded-glass material, the object is achieved according to the invention by the features of the second independent claim. Embodiments and developments of the invention, which are advantageous and are in some cases in themselves inventive, are stated in the dependent claims and in the following description.

The process according to the invention for producing granular expanded-glass material intended especially for use in a biogas plant or an anaerobic wastewater treatment plant contains the following process steps:

mixing glass powder, expansion agent and binder to form a starting mixture,
granulating the starting mixture to form granular expanded-glass material green bodies, and
foaming the granular expanded-glass material green bodies to form granular expanded-glass material particles at temperatures of from 600° C. to 950° C.

According to the invention, minerals and/or trace elements are added in the production of the granular expanded-glass material and, in this connection, especially in the production of the aforementioned starting mixture, which minerals and/or trace elements serve especially for the supply of nutrients to microorganisms used in the biogas plant or the anaerobic wastewater treatment plant.

The granular expanded-glass material according to the invention, which material serves especially for use in a biogas plant or an anaerobic wastewater treatment plant, is accordingly produced from glass powder, an expansion agent and a binder, the granular expanded-glass material being characterized by an addition of minerals and/or trace elements. The glass powder used is preferably a soda-lime glass powder, and the binder used is preferably an alkali silicate waterglass.

Here and hereinafter, minerals (also referred to as “macronutrients”) refer to those substances which are taken up by organisms, specifically by microorganisms, especially by the microorganisms used in the biogas plant or the anaerobic wastewater treatment plant, and metabolized for maintenance of life and for growth.

Preferably, but without being restricted thereto, the minerals added in the production of the granular expanded-glass material are carbon, nitrogen, phosphorus, sulfur, sodium, calcium, magnesium and/or iron.

Trace elements (also referred to as “micronutrients”) refer to those substances which are necessary for maintenance of life for organisms, again especially for the microorganisms used in the biogas plant or the anaerobic wastewater treatment plant, and occur in the organism in proportions by mass of less than 50 mg/kg. Preferably, but again without being restricted thereto, trace elements based on cobalt, manganese, molybdenum, nickel, selenium, tungsten and/or zinc are added in the production of the granular expanded-glass material.

Experiments yielded the following findings with respect to the nature and advantages of the granular expanded-glass material according to the invention (and especially with respect to the use thereof as growth support for microorganisms):

a) The granular expanded-glass material according to the invention is fundamentally suitable as support for biogas-forming and methane-forming biofilms.
b) Fermentation processes using growth supports according to the invention were always found to be superior with respect to the stability of the biogas process in comparison with control processes without growth supports.
c) Appropriately, the growth supports are broken prior to use in a fermenter/bioreactor and sieved to a defined grain-size distribution. The resulting irregular surface structure accommodates very well the formation of biofilm, i.e., the settlement of microorganisms.
d) The development of the adherent biofilm mass is considerably determined by the nature of the growth support and by the residence time in the fermenter.
e) The settlement of microorganisms on the surfaces of the growth supports is highly selective—the proportion of methanogenic organisms is distinctly higher than in the surrounding fermentation liquid or the inoculum.
f) The fusion of trace elements and minerals into the granular expanded-glass material or into the growth supports increases the biogas yield in comparison with the yield achieved with the aid of conventional growth supports.
g) Under the basic conditions in a biogas fermenter/bioreactor, the trace elements and minerals pass into solution over a long period.
h) The growth supports introduce a considerable buffering capacity into the system and thus counteract any process disturbances due to acidification.
i) A “microenvironment” which is clearly optimal for the biofilms forms on the surface of the growth supports, which microenviroment allows the archaea (i.e., the methane gas-forming microorganisms) to survive even in an acidified environment.

In a preferred process variant, the glass powder used is a ground soda-lime glass, especially having a proportion by weight of from 60% to 70%.

The binder added is preferably an alkali silicate waterglass, especially having a proportion by weight of from 15% to 25%. Advantageously, when using alkali silicate waterglass as binder with appropriate temperature control and residence time during the sintering process, it is possible to specifically achieve a desired ratio between the stability that is required and the solubility that is striven for.

It has been found to be advantageous to additionally introduce minerals and/or trace elements into the starting mixture by the expansion agent, especially by using one or more expansion agents from the following substances: dextrose, sodium nitrate, potassium nitrate, sodium carbonate, potassium carbonate, calcium carbonate and dolomite.

In a preferred embodiment, a separating agent in the form of calcium carbonate, dolomite and/or bentonite is added to the granular expanded-glass material green bodies prior to foaming for the additional supply of minerals, especially of calcium, magnesium and/or iron, to the microorganisms.

Preferably, two or more of the following minerals and trace elements with corresponding proportions by weight are used:

| | |
|---|---|
| Sodium selenite pentahydrate | 0.02000-0.04000% |
| Nickel(II) chloride hexahydrate | 0.00010-0.00030% |
| Disodium molybdate | 0.00010-0.00030% |
| Borax | 0.00010-0.00030% |
| Zinc sulfate heptahydrate | 0.00010-0.00020% |
| Cobalt(II) chloride hexahydrate | 0.00100-0.00200% |
| Copper(I) chloride | 0.00010-0.00030% |
| Potassium permanganate | 0.00020-0.00040% |

The granular expanded-glass material according to the invention, or the granular particles thereof, have in particular grain sizes which are below 2.5 mm, preferably between 0.25 and 1.5 mm.

The granular expanded-glass material according to the invention is used especially as growth support for microorganisms in a bioreactor, especially in a biogas plant or an anaerobic wastewater treatment plant.

Other features which are considered as characteristic for the invention are set forth in the appended claims.

Although the invention is illustrated and described herein as embodied in a granular pelletized glass material with trace elements, especially as growth support for selective nutrient supply of microorganisms, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is an illustration showing a scheme for an anaerobic degradation of organic substance, adapted from Weiland IP. Weiland, 2001/;

FIG. 2 is an illustration of cofactor F430 of methyl coenzyme M reductase;

FIG. 3 is an illustration showing schematically the supply and availability of micronutrients in a biogas fermenter;

FIG. 4 is an illustration showing a PCR-SSCP analysis of samples from a mesophilic biogas fermenter/LfL/;

FIG. 5 is a graph showing a feeding quantity and gas evolution in a screening experiment with different growth supports;

FIG. 6 is a graph showing the base composition and possible variations for adjusting the chemical stability in an alkali alkaline-earth borosilicate glass;

FIG. 7 is a perspective view of a (glass) syringe as miniature test fermenter for carrying out colonization experiments on different growth supports;

FIG. 8 is a perspective view of a rotary frame for storing a multiplicity of syringes as per FIG. 7;

FIG. 9 is a bar chart showing the development of the concentration of the dry biofilm over time in colonization experiments with different growth supports;

FIG. 10 is a bar chart showing the development of the microorganism population over time in colonization experiments with different growth supports;

FIG. 11 is a bar chart showing the development of the microorganism group (BAC and ARC), ARC and the % proportion thereof of the total population over time in colonization experiments with different growth supports; and

FIG. 12 is a bar chart showing the concentration of the dry biofilm and the biogas yield in colonization experiments with different growth supports.

### DETAILED DESCRIPTION OF THE INVENTION

Parts and steps that correspond to one another are always provided with the same reference signs in all the figures.

### 1. BACKGROUND INFORMATION RELEVANT TO THE INVENTION

First of all, some microbiological, biochemical and process-related aspects concerning the invention will be explained below for the purposes of elucidating the invention.

a) The Biogas Process

Fundamentals of Biogas Formation

The anaerobic conversion of biomass and thus the obtaining of the renewable energy source methane is one of the currently most efficient processes for producing renewable energy sources from biomass ISRU, 2007/.

Building on energy production by means of photosynthesis, macromolecules are synthesized by the plants as metabolic output. From a stoichiometric point of view, the macromolecules can, in the case of anaerobic degradation in biogas plants, be converted into methane and carbon dioxide with a very high efficiency.

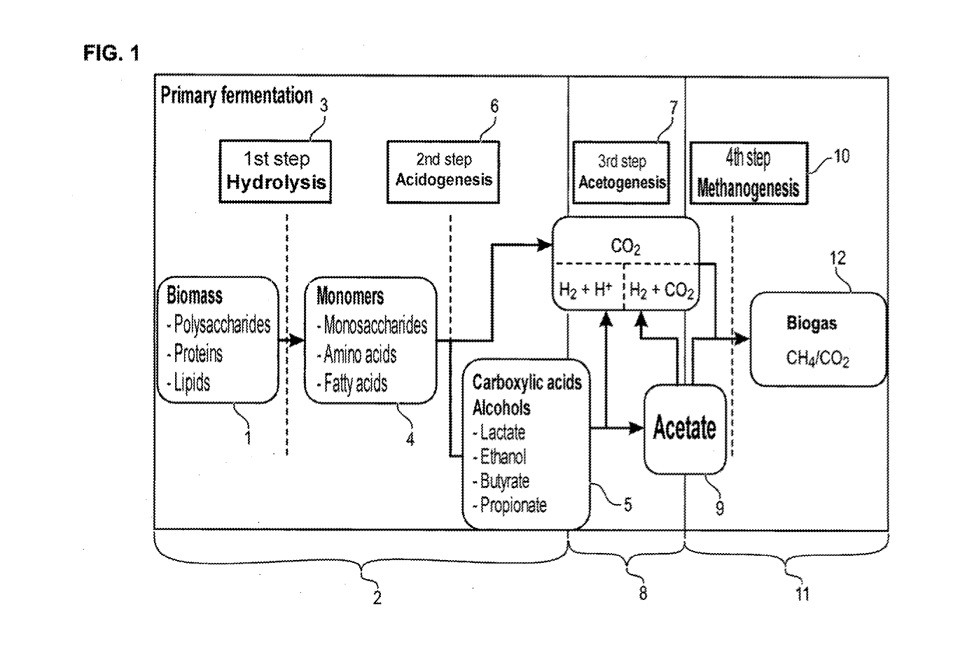

This conversion is depicted schematically in FIG. 1.

The degradation of organic substance (i.e., biomass 1, for example polysaccharides, proteins, lipids) under exclusion of air is always initiated by the so-called primary fermentation 2 (identified in FIG. 1 as a gray box). The fermentation itself is a microbial conversion process for the purposes of energy production. However, in this energy metabolism, neither oxygen nor some other external electron acceptor (e.g., NO₃⁻) is involved.

The primary fermenters secrete exoenzymes, which break up the complex macromolecules of the starting materials through the separation of the hydrogen bonds with simultaneous incorporation of water. This step is called hydrolysis 3 and allows the microorganisms to take up and to convert the monomers 4 or dimers that are formed.

The individual building blocks (monomers 4) taken up by the primary fermenters are converted by the microorganisms to form building blocks 5, specifically organic acids, alcohols and carbon dioxide (FIG. 1, 2nd step) acidogenesis 6.

In the third conversion step, acetogenesis 7, the acids and alcohols that are formed are converted to form acetic acid (acetate 9), hydrogen and carbon dioxide by the secondary fermentation 8.

The fourth conversion step, methanogenesis 10, proceeds only under strict exclusion of air (identified in FIG. 1 as anaerobic respiration 11). In this stage, the methanogenic microorganisms form, from acetic acid or from hydrogen and carbon dioxide, the methane and carbon dioxide that are present in the biogas 12.

Whereas the direct conversion of acetic acid into methane and carbon dioxide is a simple disproportionation, the formation of methane from carbon dioxide and hydrogen is, from a biochemical point of view, a process of anaerobic respiration. This is understood to mean the oxidation of organic compounds under exclusion of air, in which oxidation oxidized organic or inorganic compounds act as electron acceptor instead of oxygen/Klocke et al., 2009/.

### 1.2. Bioreactor/Fermenter

Bioreactors, frequently also referred to as fermenters, are vessels in which certain microorganisms, cells or small plants are cultivated (fermented) under conditions that are as optimal as possible.

Important factors which are regulatable or controllable in the majority of bioreactors are the composition of the culture medium (nutrient solution or substrate), the supply of oxygen or leak-tightness with respect to entry of oxygen, temperature, pH, sterility and other factors. The purpose of cultivation in a bioreactor can be to obtain the cells or constituents of the cells or to obtain metabolic products. The degradation of chemical compounds can, too, take place in bioreactors, such as, for example, in the case of wastewater treatment in wastewater treatment plants.

In bioreactors, very different organisms are cultivated for different purposes. Therefore, multiple reactor variants of differing design are available. Typically, stirrer-tank reactors are made of metal, but greatly differing variants, such as, for example, fixed-bed reactors, photobioreactors, etc., are also used.

Bioreactors are used in very different sectors in the process industry, such as, for example, in:

Wastewater treatment plants, containing process stages of a biological nature. In the case of the activated sludge process, there is initially an aerobic step in which dissolved compounds are bound by microorganisms in the form of the biomass that is formed.

A further biological-process stage is anaerobic wastewater treatment; it serves, under very high COD load, for the removal of harmful or disruptive organic carbon compounds by means of microbiological degradation processes which proceed in the absence of oxygen (anaerobically). In the process, bacteria obtain the energy required for their metabolism from the conversion of the organic carbon compounds to form organic acids and, subsequently, mainly to form methane, carbon dioxide, hydrocarbon.

In the case of the anaerobic system, only about 0.1 kg of dry sludge material are formed from 1 kg of COD load; the remaining carbon is, to an extent of roughly 90%, converted into methane, CO₂ and H₂O.

In the case of the aerobic system, roughly 0.5 kg of dry sludge matter are formed from 1 kg of COD load; roughly 50% is converted to form CO₂ and H₂O.

The biomass from the activated sludge process (aerobic system) that is formed can be converted to form methane-rich wastewater-treatment gas via an anaerobic process step in an additional fermenter (digestion tower).

Biogas Plants

The biomass 1 used is degraded in an anaerobic process comprising multiple steps (hydrolysis 3, acidogenesis 6, acetogenesis 7 and methanogenesis 10) to form biogas 12 and fermentation residue (see FIG. 1). The vessels are closed off in an airtight manner and generally have a stirrer. In order to be able to operate biogas reactors in an optimal manner, it is necessary to know what biological and process-related conditions are prevailing in the fermenter and what the nutrient status thereof is. But on the majority of plants, only a few parameters, such as pH and gas quality, are measurable. In many cases, there are not even any data relating to methane or carbon dioxide contents in the gas. Frequently, the operators operate their plants on the basis of experience.

Breweries and Wineries

Breweries or wineries use bioreactors, too, though the bioreactors are referred to here as fermenting vats for example. Here, the microorganisms used are yeasts which convert the sugar from the mash or the grape juice into alcohol and carbon dioxide (CO₂).

Pharmaceutical Industry and Cosmetics Industry

The most valuable bioreactor-produced products are medical/pharmacological products such as, for example, erythropoietin (EPO), which has become known as a doping agent, or modern insulins.

The biological activity of microorganisms in bioreactors is determined by their metabolism. The better the microorganisms convert organic substances, the higher the biological-process yields. If the process-related parameters in the reactor are not optimal or nutrients are missing, the biological activity of the microorganisms drops.

### 1.3. Growth Rates of Microorganisms

Microorganisms can multiply at differing rates. Their specific metabolic output and thus the conversion of the substrate depend greatly on favorable environmental conditions, on the presence of competitors/robbers, and on the energy which the reaction provides them. The denser the population and the higher the substrate availability, the more rapid the substrate conversion—though only within certain limits, since other factors may have a limiting effect.

The growth of hydrolytic and acidogenic microorganisms is, to the extent it has so far been possible to test this, on average more rapid than that of syntrophic bacteria and of methanogenic archaea. The syntrophs and methanogens are thus usually the bottleneck in biogas production. They are the “weakest” biocenosis member, by which the overall process has to be determined.

Therefore, when starting up a fermenter, it is sensible to increase the addition of substrate slowly and to provide a relatively long residence time in order to give all members of the biogas biocenosis sufficient time to establish an efficient population density. If too much substrate is added at once, there is growth of the hydrolytic/acidogenic microorganisms in particular and the acids which arise cannot be completely converted to form biogas. As a result, the pH in the fermenter drops.

Since methanogenic archaea react more sensitively to acid than hydrolytic/acidogenic bacteria, the proportion of organic acids in the fermenter rises increasingly and can lead to a crash when the loading rate is not distinctly reduced or the feeding is suspended. Suspension of the feeding lowers the formation of acid and leads, if this is done in a timely manner, to a recovery of the process. A supporting measure can be to increase the pH by addition of substances which act as a base (e.g., sodium hydrogencarbonate, lime, burnt lime/slaked lime).

For the same reasons, sudden changes during operation affect particularly the syntrophs and methanogens. They need the longest to adapt themselves to the new circumstances. The result is usually an acidification as outlined above.

Thus, it is necessary to proceed carefully when, for example, changing the substrate or changing the operating temperature. If operation was stable prior to the change, the loading rate with the new substrate should be initially low and the residence time should be kept long. In this way, the weak members of the chain get sufficient time to establish an effective population which is adapted to the new conditions and which is of a sufficient density. After this newly assembled biocenosis has stabilized (typically after about 20 days), the loading rate can be increased slowly. If the loading rate is increased too rapidly, the association of syntrophs/methanogens cannot regrow rapidly enough. A further approach is to ensure that the concentration of the syntrophic/methanogenic organisms is kept artificially high by process-related means, for example via recycling of fermentation residue or immobilization.

On the basis of general experience, the limit is, for example for corn silage and optimal supply of trace elements, in the range of 6-10 kg ODM/(m³*d).

### 1.4 Minerals and Trace Elements

### Definition of Terms and Meaning

Minerals are inorganic nutrients essential to life that cannot be produced by the organism itself; they must be supplied to said organism in the diet. Since the minerals are usually present in the form of inorganic compounds, they are, unlike some vitamins, insensitive to the majority of preparation methods. For example, they cannot be destroyed by heat or air. For human, animal and plant organisms, minerals are important inorganic compounds. In this connection, they do not serve for the provision of energy, but instead act as building material and active substance and thus assume important regulatory functions in the organism. “Sie sind als Katalysatoren am Stoffwechsel beteiligt, unentbehrlich für die Regulation des pH-Werts, [ . . . ], für die Schaffung von Puffersystemen, [ . . . ], oder die Förderung von Enzymsystemen” [They are Involved in Metabolism as Catalysts, Indispensible for pH Regulation, [ . . . ], for Establishing Buffer Systems, [ . . . ], or Supporting Enzyme Systems]/Ternes et al., 2005/.

Minerals are taken up by plants from the soil via the roots and incorporated in their biomass. They cannot be incinerated and therefore remain as unincinerated residue in the ash. This property can be utilized for the determination of the proportion of minerals in the plant material or in the fermenter sludge.

Out of the altogether 92 different natural elements, 40-50 can be detected in plants. However, only 16 thereof are indispensible for plants. These nutrients are therefore also referred to as essential nutrients.

The minerals in an organism are classified according to two dimensions, according to concentration or according to function.

Concentration: Elements in relatively high concentrations in the organism—over 50 mg per kg of body weight—are referred to as macroelements (minerals). Elements at less than 50 mg per kg of body weight are called trace elements or microelements/Entrup et al., 2008/. For a range of trace elements, it has still not yet been clarified whether they are a coincidental constituent of the organism in question or whether they actually have a physiological function.

Function: On the basis of their function in the organism, a distinction is made between building materials (minerals) and regulator substances (trace elements).

A comparison of numerous biogas plants involving similar technology, virtually identical substrates and the same mode of operation, which comparison was carried out by the University of Hohenheim, showed that the plants greatly varied especially in the maximally achievable volume-specific output.

In the case of individual biogas plants, process disturbances already occurred once the (organic dry matter-based) digester loading rate was increased to above 2 kg per cubic meter per day (ODM=2 kg/(m³*d)). By contrast, in other biogas plants, it was possible to achieve more than twice as high digester loading rates of, in some cases, over 5 kg/(m³*d) (ODM) without any problems.

Studies of “less efficient” fermentation substrates from operational plants, which studies were carried out in the biogas laboratory of the University of Hohenheim, showed that the limited efficiency of the fermentation biology was due to the “deficient nutrition” thereof. By addition of previously deficient trace elements, the productivity of the bacteria was distincty increased, with result that it was possible, within the same period with unchanged fermentation volume, for distinctly more biomass to be converted to form methane. Process inhibition with accompanying acid enrichment occurred.

Macronutrients/Minerals

For their metabolism and for the building of their cellular material, the microorganisms involved in the biogas process require various minerals. In this connection, the quantities required are species-specific. “Die Trockenmasse von Mikroorganismen besteht etwa zu 50% aus Kohlenstoff, zu 11% aus Stickstoff, zu 2% aus Phosphor and zu 1% aus Schwefel” [The Dry Mass of Microorganisms Consists of about 50% Carbon, 11% Nitrogen, 2% Phosphorus and 1% Sulfur].

It can be inferred therefrom that carbon is the most important nutrient, followed by nitrogen, phosphorus and various sulfur compounds/Bischofsberger et al., 2005/.

Carbon is, after water, the main constituent in microorganisms. The substrate supplied serves substantially as the carbon source.

Nitrogen is, after carbon, the nutrient that is required the most. It is required in particular for protein biosynthesis, i.e., for the formation of enzymes which carry out the reactions in metabolism. However, excessively high nitrogen contents in the substrate can lead to an inhibition of the microbial activity in the fermenter.

Phosphorus is involved in the formation of the energy carriers adenosine triphosphate (ATP) and nicotinamide adenine dinucleotide phosphate (NADP) in microorganism metabolism. Phosphate deficiency therefore leads to waning of metabolism.

Sulfur is a constituent of the amino acids cysteine and histidine and thus essential for the formation of important enzymes in metabolism.

Other sulfur compounds too play a crucial role in the metabolic cycle, for example FeS complexes as redox partners in electron transport.

Sodium is important for (some) archaea, since the energy carrier ATP is produced not only via the performance of “proton pumps”, but also via “Na⁺ pumps” in the case of said archaea/Deppenmeier et al., 1999/. In the case of undersupply of Na⁺, energy metabolism in methanogens and thus methanogenesis in biogas production may thus collapse. This may already be the case when 50 mg of Na⁺ per g of dry matter is fallen short of.

In addition to C, N, P, S and Na⁺, calcium, magnesium and iron also perform important functions in metabolism in the area of minerals. Calcium and magnesium are important structural elements, for example for enzymes.

Iron undertakes three important tasks in the biogas process. Firstly, iron is a binding element in sulfide precipitation. Owing to iron binding sulfur, iron sulfide can be precipitated, the result being that sulfide toxicity in the fermenter is reduced.

Secondly, iron serves as reaction partner in microbial metabolism.

Methanogenic bacteria require a transport system for the reduction of CO₂ to form CH₄. Through the reduction of Fe³⁺ to form Fe²⁺, iron acts at first as electron acceptor. As a result, Fe²⁺ can be utilized at a later time as electron donor and thus as energy carrier. Lastly, iron is also additionally a constituent of many enzymes.

Fundamentally, it is not only the quantity of a nutrient, but also the optimal ratio of all the nutrients to one another that counts for obtaining optimal process conditions. To this end, a C/N/P ratio of approximately between 100:5:1 and 200:5:1 has been recommended/Effenberger et al., 2007/.

Micronutrients/Trace Elements

Owing to their low concentration in the biomass, micronutrients are also referred to as trace elements. Trace elements reach the soil as a result of physical and chemical weathering of rocks. Consequently, the trace-element content in the soil varies depending on the source rock, the climate and the influence of planting or of farming.

Plants require trace elements in order to survive. They extract these substances from the soil, with, however, only a few grams per hectare being involved. The micronutrient content in plant biomass is correspondingly low.

A sufficient presence and availability of some trace elements is necessary for life for microorganisms. Methanogenic archaea require the elements cobalt (Co), manganese (Mn), molybdenum (Mo), nickel (Ni), selenium (Se), tungsten (W) and zinc (Zn).

Cobalt serves especially as central atom in corrinoids and vitamin B12 enzymes. In this connection, cobalt can be incorporated as central ion in three different forms: as Co³⁺, Co²⁺ or Co⁺. Cobalt-containing enzymes are present both in methanogenic and in acetogenic bacteria.

Manganese (Mn) has, in the case of anaerobic organisms, similar properties or functions as iron. Through the reduction of Mn⁴⁺ to form Mn²⁺ and of Fe³⁺ to form Fe²⁺, they utilize these trace nutrients as oxidizing agent. Through later oxidation reactions, electrons can in turn be provided for further reactions.

Nickel is a kind of universal element in the biogas process, since it is involved in the building of many different enzymes. For instance, there is a central nickel atom in, for example, urease, hydrogenase, cofactor F430 and additionally many other enzymes.

Urease brings about the hydrolysis of urea. The ammonia released in the hydrolysis serves as nitrogen source for many microorganisms.

Hydrogenase catalyzes the oxidation-reduction reaction of hydrogen and, in this connection, plays an essential role.

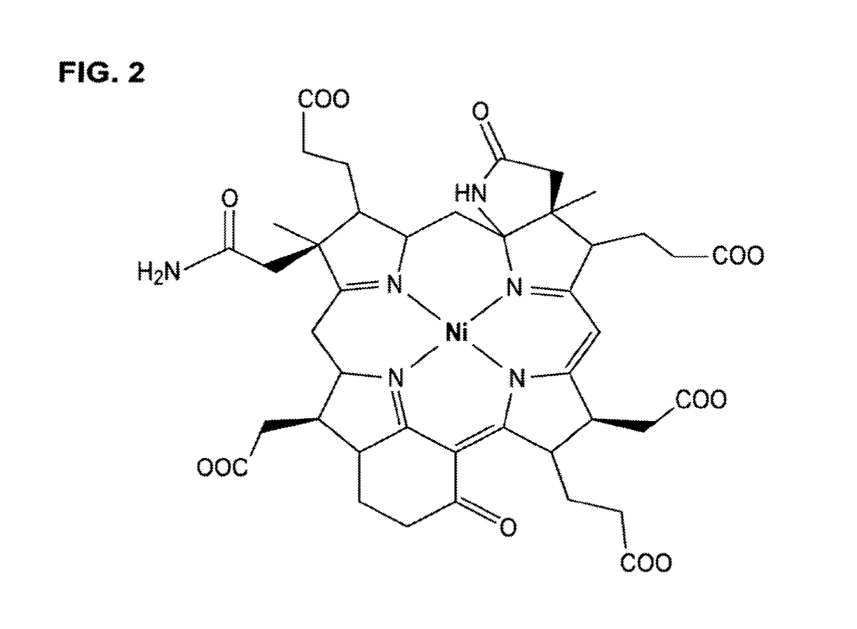

Cofactor F430 (see FIG. 2) is indispensible for the last step of methane formation. This cofactor has a central nickel atom.

Molybdenum (Mo) and tungsten (W) have similar chemical properties and also undertake comparable tasks during anaerobic conversion. For instance, they usually catalyze the oxidation and reduction reactions of CO₂. In addition, studies have shown that, in some cases, molybdenum can be replaced by tungsten. Molybdenum is thus the only known example in which a central atom of an enzyme can be replaced by another without loss of the enzyme-specific action/Bertram et al., 1994/.

Selenium is important especially for the building of proteins such as, for example, selenocysteine or selenomethionine. Some methanogenic archaea require the selenium-containing proteins for the oxidation of hydrogen. For the archaea, an inadequate selenium concentration can therefore also become the growth-limiting factor/Chasteen, Bentley, 2003/.

The importance of other elements (e.g., copper, aluminum, vanadium and boron) for biogas production is still unclear. However the heavy metals copper, silver, lead, mercury, cadmium, gold and arsenic are typically toxic.

For all trace elements, the concentration in which the elements are present and the availability thereof for the microorganisms is of crucial importance/Ch. Bauer, M. Lebuhn, A. Gronauer; 2009/. For each component, there is both a kind of minimal concentration and a maximal concentration, with the microbial metabolic process being limited or inhibited when they are, respectively, fallen short of or exceeded/Bischofsberger et al., 2005/.

If the trace elements are present in excessively low concentrations, it is possible, inter alia, that the enzymes and coenzymes required for metabolism are no longer sufficiently formed. As a consequence, the efficiency of the methanogenic microorganisms drops. The primary fermentation, i.e., the formation of the longer-chain carboxylic acids and alcohols, is not affected thereby. As a result, there is an enrichment of the metabolic products formed in these upstream process steps—especially propionic acid.

Studies show that the nutrient contents in biogas plants fluctuate within a broad range. The fermentation substrate analyses of over 700 different biogas plants show that the contents of individual elements can fluctuate by a factor of 200 between the lowest and the highest values (Table 1).

TABLE 1
Mineral contents in the fermentation substrate of biogas
plants, data from 700 analyses/Lindorfer, 2009/

| | | | | Iron | |
|---|---|---|---|---|---|
| | Sodium | Sulfur | Copper | mg/kg | Molybdenum |
| | g/kg DM | g/kg DM | mg/kg DM | DM | mg/kg DM |
| Minimum | 0.37 | 0.5 | 7.3 | 280 | 0.26 |
| Maximum | 75.3 | 15.8 | 501 | 20166 | 9.53 |
| Mean | 4.5 | 1.84 | 52.24 | 2413 | 2.6 |
| Factor | 204 | 32 | 69 | 72 | 37 |

If a trace element deficiency is established in the fermentation substrate of a fermenter and said deficiency is compensated for by the addition of industrial mineral mixtures on a plant-specific basis, a distinct process stabilization with accompanying acid degradation usually begins within a few days and the digester loading rate can be increased again or increased further.

Minerals and Trace Elements in the Biogas Fermenter

Building on the findings from the ongoing experiments, the supply of nutrients and trace elements to the methane-forming biocenosis is increasingly coming under focus. Until about 5 years ago, there was in practice generally a lack of knowledge about the more precise needs/Effenberger M., Lebuhn M., Gronauer A.; 2007/. Since then, much has been researched, published and patented concerning supplementation/Haun, 2008, Hölker, 2010, Agraferm, 2009/. In this connection, the required quantity of individual elements, for example for enzyme metabolism, is only one of the many factors for biogas yield.

A multiplicity of different suppliers for so-called “standard trace-element mixtures” are already on the market. It is difficult for the plant operator to have a clear view of the diverse offerings and the correct dosage thereof. However, it is important for operators of biogas plants to note in this connection that “much more” does not bring much more. On the contrary, an overdosage can have an inhibitory or toxic effect on the biocenosis. Not to mention the effects on fermentation residue quality and on bioavailability in the soil, which can lead to conflicts with, for example, the Düngemittelverordnung [fertilizer regulation]. This has to be increasingly taken into account in the future.

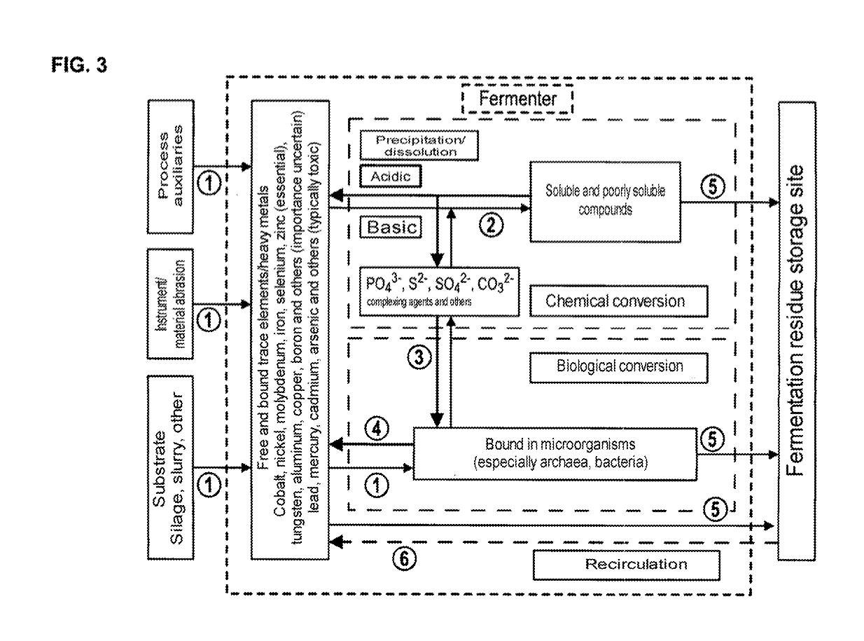

Through the added substrate, via instrument abrasion and through process auxiliaries, trace elements and other heavy metals reach the biogas fermenter in a form which is bound and is frequently not available biologically (FIG. 3). By means of physical (e.g., temperature, friction, comminution), chemical (e.g., pH) and biological (e.g., microbial degradation) processes, they are dissolved and thus become microbially available.

(FIG. 3 (1)). Whereas acidic conditions promote their solubility, they are increasingly converted into poorly soluble compounds at relatively high pH in the presence of free phosphate, sulfide, sulfate and/or carbonate. They are thus at first withdrawn from direct access by microorganisms (2). However, some microorganisms can, via outward transfer of complexing agents, “capture” unavailable trace elements and utilize them for themselves (3). Following the death of microorganisms, trace elements in bound and dissolved form are re-released (4) and can be used again in the internal nutrient cycle. Trace elements are outputted with the fermenter content and the biomass into the fermentation-residue storage site (5). In the event of a recirculation of the fermentation residue, the trace elements and heavy metals that are carried along are available again in the fermenter for the microorganisms (6).

In biogas plants, there is the risk of situations of trace element deficiency, especially in mono-renewable resource operation; however, there are also such reports in the case of plants with addition of liquid manure. A typical consequence of trace element deficiency is the inhibition of methanogenesis and an associated acidification.

In appropriately simulated experiments with corn mono-operation, it was possible to detect only hydrogenotrophic, but no acetoclastic, methanogens in the acidified fermenters/Lebuhn et al., 2008b/. In this connection, the loading rate prior to the acidification was relatively low, and this is why the presence of acetoclastic methanogens, while expected, was not found. Clearly, hydrogenotrophic methanogens react less sensitively to a trace element deficiency with acidification than acetoclastic methanogens.

According to studies/Lebuhn und Gronauer, 2009/a content of about 50 μg of Co per L (approximately 750 μg of Co per kg of DM) appears to be a sensible order of magnitude for stable operation of a renewable resource plant. For selenium, the corresponding concentrations are about 5-times lower (about 10 μg of Se per L, or approximately 150 μg of Se per kg of DM), but higher by about a factor of 10 for Mo and by about a factor of 40 for Ni.

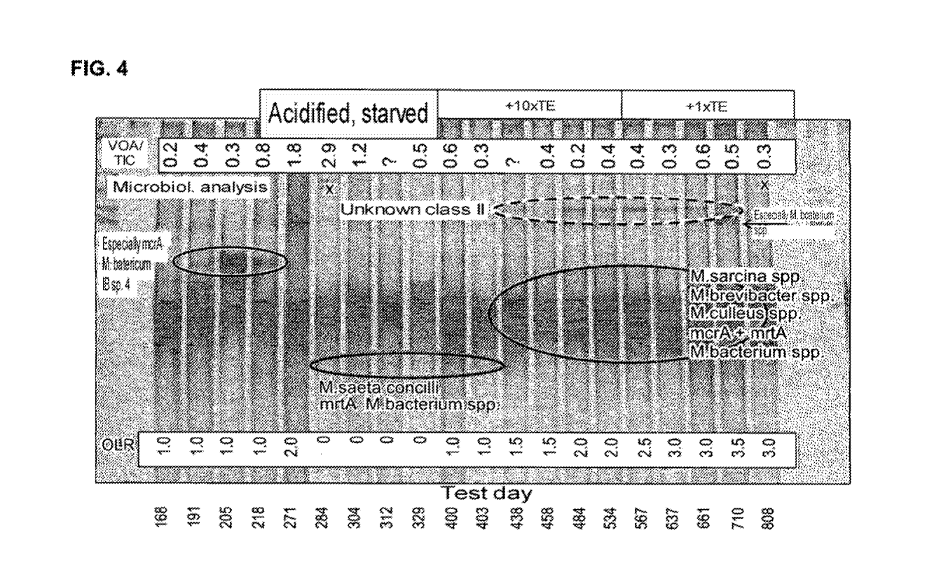

FIG. 4 shows the activating effect of the trace element additive for the methanogenic archaea. It shows a PCR-SSCP analysis of various samples from a mesophilic test fermenter fed only with corn silage, from the Institut für Landtechnik und Tierhaltung [Institute of Agricultural Engineering and Animal Husbandry] of the Bavarian State Research Center for Agriculture [LfL]. In the analysis, a portion of the DNA of the key enzyme in methanogenesis (mcrA/mrtA) was multiplied and resolved. The resolved bands can thus be assigned solely to methanogenic archaea. There were distinct changes in the pattern of bands depending on the mode of operation and on the state of the methanogenic population. For example, the diversity and the number of methanogens decreased during the acidification of the fermenter. By contrast, during the recovery brought about by the addition of trace elements, new bands representing the growth of certain methanogenic archaea appeared.

### 2. GRANULAR EXPANDED-GLASS MATERIAL AS GROWTH SUPPORT FOR MICROORGANISMS

### 2.1 Preliminary Experiments with Modified and New Material Types

Procedure for the Preliminary Experiments

To create uniform ambient conditions, a screening experiment was carried out in a pilot plant/climate chamber at PORAVER. To this end, 10 test fermenters, which were substantially formed by closed plastic containers, were constructed with an appropriate environment and loaded with the material types listed below.

TABLE 2
Material types for further experiments at Dennert Poraver
[DP]

| | |
|---|---|
| Type 1 | round spray grain |
| Type 2 | broken grain |
| Type 3 | broken grain with xanthan gum coating |
| Type 4 | broken grain with xanthan gum/nutrient |
| | solution coating |
| Type 5 | broken grain with 3rd coating (version from |
| | Sudchemie) |
| Type 6 | broken grain with zeolite coating |
| Type 7 | reductively melted grain |
| Type 8 | dolomite as expansion agent |
| Type 9 | granular iron oxide (pure/unexpanded) |
| Type 10 | magnetic PORAVER (magnetic separator prior |
| | to loading) |

Up to number 6, the modifications are modifications to the test grain already used at the Leibniz Institute for Agricultural Engineering and Bioeconomy [ATB]. With the same formulation, only the production method was shifted to the mixed granulation and the grain broken. The respective coatings were subsequently applied.

Materials 7 and 8 are newly developed materials having modified chemical properties.

Types 9 and 10 were used to secure evidence of the effectiveness of the growth supports according to the invention.

The culture broth was prepared by using inoculum from the test setup at the ATB, ruminal contents from Schlachthof Nürnberg and glass breakage from the glass heap in PORAVER production. The glass breakage was added, since it has been known for a long time that storage of the processed recycled glass for several months leads to composting and, at the same time, organic contaminants and adhesions are degraded. Since there are definitely anaerobic conditions in the interior of the glass heap, it was assumed that a biocenosis developed in this environment, which biocenosis should be well adapted to a glass substrate.

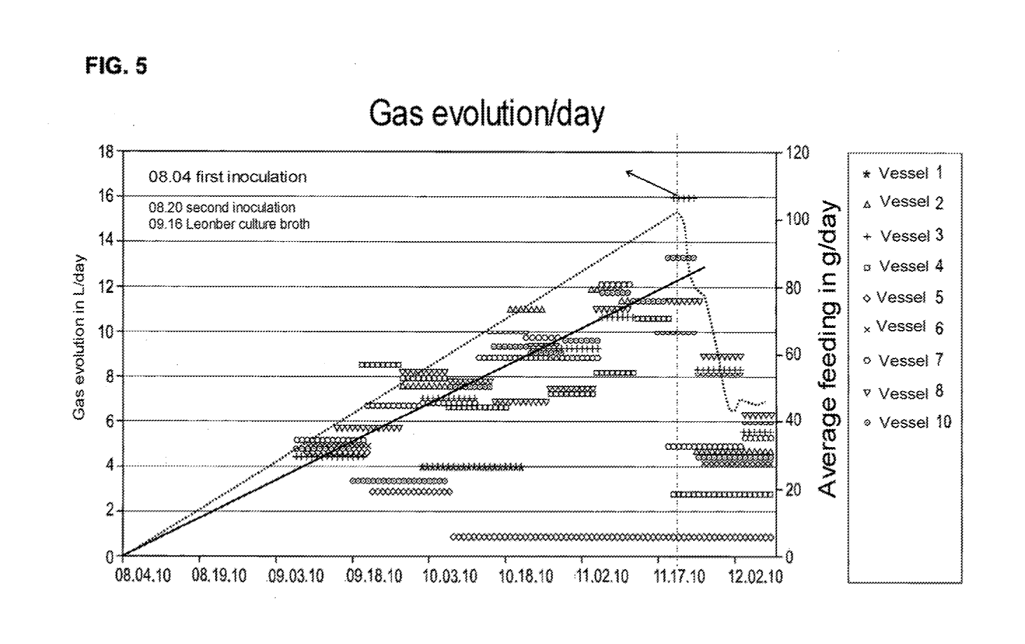

Through continuous monitoring and measurement of the pH values, of the VOA/TIC values and of the gas-formation rates of the individual fermenters, it was possible to very rapidly establish differences in the behavior of individual fermenters (FIG. 5).

Assessment of the Results of the Preliminary Experiments

Studies using phase-contrast microscopy to establish the colonization density yielded very encouraging results for a few test types. However, the results are not shown here owing to the poor presentability.

Type 1, already in use in the project at ATB Potsdam (though with prewashed surface), was colonized distinctly more rapidly than in the preliminary project, but performed distinctly poorer, in the colonization and in the gas formation, than all the other variants in the test. This result indicates that a smooth surface is, for physical reasons, an obstacle to good colonization, even though chemically the environmental conditions are the same as in the case of the other material types.

In the case of the fermenter with the granular iron oxide, type 9, acidification already occurred at low loading rate and after the shortest time, and this led to the early elimination of this type from the test series.

Magnetic PORAVER, type 10, is of only limited suitability as growth support because of the very slow and deficient colonization due to its excessively smooth surface nature.

The beneficial influence of a coating with xanthan gum (material type 3 and 4) on the colonization of the growth supports was to be expected. This approach is generally known in biotechnology and can be considered to be prior art.

However, the results for type 4 were of particular interest. In the case of this variant, it was possible to observe a very rapid and intense colonization with above-average biogas formation. This evidently proves the positive effect of nutrients on the vitality of the fermenter biocenosis.

However, as the test period advanced, the difference in relation to the other broken grain types became ever smaller and was no longer determinable after a few weeks. It can be deduced therefrom that the doping of the surface coating with nutrients exhibits a positive effect in the colonization. Very obviously, the supply of trace elements caused the microorganisms to preferentially settle in this environment and to react with increased metabolic activity. However, as organic compound, xanthan gum is dissolved or degraded in a very short time in the metabolic processes in the fermenter.

If a coating is no longer present, then, here too, it is only the glass surface and the interaction thereof with the colonizing microorganisms that is crucial for further use, as in the case of the other material types.

Broken grain type 2 as well as the material from the reductively melted series, type 7, in which additionally the cheaper and easier-to-process magnetite had been used instead of the γ-Fe₂O₃, showed a very intense bacterial colonization after a relatively slow start. In particular, methanogenic archaea of the species Methanosarcina were detectable.

With dolomite as expansion agent, type 8, a very strong and dense colonization with microorganisms likewise developed after a delay at the start of the test period.

The initial difficulties in the colonization can be attributed to the fact that, in the case of the calcined dolomite (for the reactions, see under section 2.2.3.) on the surface of the granular material, the high pH of 12.6 first had to be reduced. This high alkalinity ensues owing to the reaction of calcined dolomite upon introduction into aqueous solution. After the high pH was buffered by the organic acids from the substrate, the organisms found appropriate ambient conditions which caused them to colonize.

From the comparison with the other material types with regard to colonization density and gas-formation rate, it can be deduced that the colonization of the growth supports is obviously promoted in the presence of calcium and magnesium in readily soluble form.

The influence of the unconverted calcium/magnesium carbonate as buffer for pH stabilization in the substrate is still to be studied.

In summary, it can be stated that:

1. Soda-lime glasses are highly suitable as growth substrate for the immobilization of the fermenter biocenosis.
2. Broken grain is preferentially colonized, since shear-off and strip-off of the biofilms due to movement (stirrers) is prevented owing to the structure-rich surface.
3. Chemical and physical properties of the glass appear to have effects on the vitality of the biocenoses.
4. The application of minerals and/or trace elements in the expansion process and/or the admixing thereof in the formulation showed positive effects. The minerals or trace elements can be present in the form of oxides, carbonates and hydrates.
5. Coatings with organic substrates have a positive influence, but are only necessary when a very rapid colonization is striven for.

### 2.2 Further-Developed Granular Expanded-Glass Material as Growth Support

### 2.2.1 Glass as Basis of the Substrate Material

Alkali alkaline-earth silicate glasses are the oldest glass type produced artificially by humans. These include the flat glasses (window glass) and packaging glasses that are melted in large quantities.

Borosilicate glasses are glasses which are highly chemical-stable and temperature-stable and which are used especially for glass instruments in the laboratory, in chemical engineering and in households. The good chemical stability with respect to water, many chemicals and pharmaceutical products (hydrolytic class 1) is explained by the boron content of the glasses.

When glasses are exposed to an aqueous medium, the near-surface alkalis and alkaline earths first go into solution. Owing to this process, the structure is weakened and the network-forming SiO₂ is likewise dissolved. The oxides going into solution or the reaction products thereof are, with the exception of the SiO₂, found again in the list of minerals and trace elements (section 1.4).

However, the typical utility glasses can be classified as very stable in terms of their chemical stability (hydrolytic class 1 or 2). The expected dissolved quantity of Na, K, Ca and Mg ions is so low that, under normal conditions, an adequate and specific supply of nutrients to the microorganisms cannot be assumed.

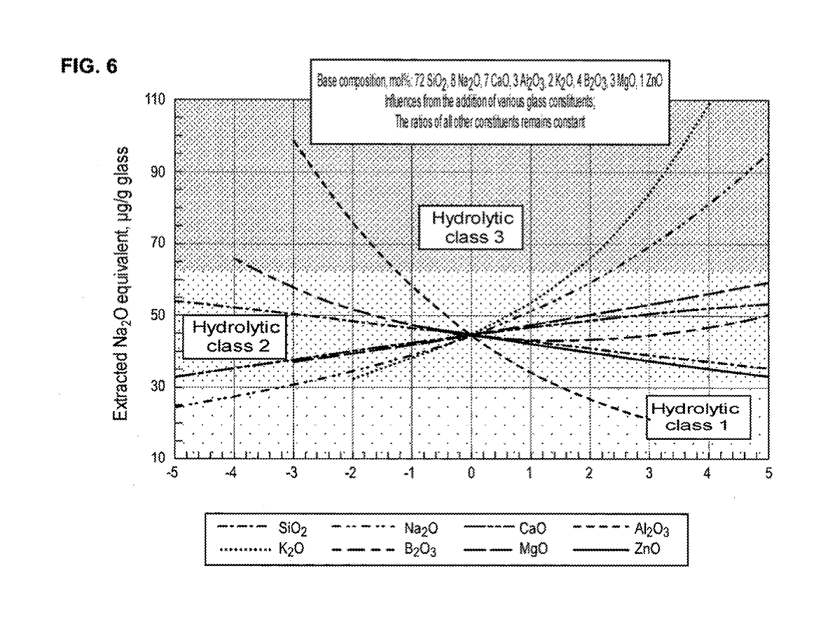

As depicted by way of example in FIG. 6, it is fundamentally possible, through alteration in the glass batch, to melt a glass with relatively high solubility (hydrolytic class 3). The strongest influence on chemical stability is achieved by an increase in the alkali proportions (Na₂O, K₂O). But also through the increase in the alkaline earth proportions (CaO, MgO), though only within the limits defined by glass technology, a distinct effect can be achieved.

However, this approach would require, in the case of the growth supports, having to melt their own glass. For the intended application, this is out of the question for economic reasons. However, if use in the food technology sector should be envisaged over the course of further development, it will not be possible, for reasons of product safety and traceability, to avoid using a self-produced glass.

### 2.2.2 Waterglass for Controlling Solubility

The green body used to produce the granular expanded-glass material is mainly composed of glass powder, iron oxide and waterglass. The addition of different quantities of waterglass opens up a very good possibility of specifically adjusting the chemical stability of the overall system in aqueous media and of greatly lowering it in comparison with base glass. If an alkali silicate glass (waterglass) is added to the ground base glass (soda-lime glass), the result of this, depending on the quantity added, is not only a lowering of the sintering temperature, but also a lowering of the chemical stability. Waterglass is chemically less stable owing to its composition of only two components—the network former SiO₂ and a network modifier (flux), in this case Na₂O—especially at high alkali proportions. With appropriate temperature control and residence time during the sintering process, homogenization (oxide equalization) between the base glass (glass powder) and the waterglass is prevented. Therefore, it is possible to achieve a desired ratio between the stability (mechanical and chemical) that is required and the solubility that is striven for.

### 2.2.3 Separating Agents as Additional Mineral Providers

The use of a separating agent is required in order to prevent the granular material from sticking together under the action of heat in the expansion process. It is an inert substance, which is to be chosen such that there are no interactions or only very low interactions with the granular glass material under the respective process conditions. In most cases, the substances are substances of mineral origin such as, for example, sand, aluminum oxide, various carbonates, but also high-firing clays, specifically in the form of powders and dusts.

For the doping of the growth support with the minerals calcium, magnesium and iron, particular attention is paid to limestone, dolomite and bentonite.

Dolomite is a carbonatic calcium magnesium mineral, CaMg(CO₃)₂; it is rock-forming in rock of the same name, in dolomitic limestone and in various sedimentary rocks. In addition to ankerite, CaFe(CO₃)₂, dolomite frequently occurs as a hydrothermal gangue. Its color can range from white, gray, yellowish to red-brown. Under the action of heat, dolomite starts to decompose from approximately 830° C. according to the following reaction:

CaMg(CO₃)₂+Temp.→CaO+MgO+2CO₂

The reaction products CaO, MgO, but also unconverted CaMg(CO₃)₂, attach by adhesion or van der Waals forces to the surface of the foamed granular material.

If they are introduced into an aqueous solution, CaO/MgO act as acid buffer and are available as hydroxides to the microorganisms very rapidly for metabolism:

CaO+MgO+2H₂O→Ca(OH)₂+Mg(OH)₂.

In the case of a saturated solution (1.7 g/L or 0.009 g/L), the pH is around 12.

Bentonite is a mixture of various clay minerals, containing montmorillonite (60-80%) as the most important constituent; this also explains its strong water-uptake capacity and swelling capacity. Further accompanying minerals are quartz, mica, feldspar, pyrite or else calcite. It is formed mainly by weathering from volanic ash.

### 2.2.4 Growth Supports Containing Trace Elements and Minerals

Trace Elements Added

Building on the results from the previously conducted experiments, current results from biogas research in relation to the supply of nutrients to microorganisms, a new growth support was developed.

The base composition corresponds to the two material types 7 and 8 of the preliminary experiments. The nature and the quantity of the trace elements added were compiled on the basis of a dissertation relating to Methanosarcina mazei substrate conversion/Krätzer 2011/and work on the same topic at the LfL in Freising/Bauer, Lebuhn, Gronauer; 2009/.

In a first variant for producing granular expanded-glass material containing trace elements, a starting mixture composed of premixture one and premixture two according to the following composition was mixed.

Premixture One

| | |
|---|---|
| Iron oxide 316 | 14.3764% |
| Glass powder series | 64.3987% |

Premixture Two

| | | | |
|---|---|---|---|
| Dextrose C-DEX 02001 | 2.1663% | | |
| Waterglass | 19.0242% | | |
| Water, hot | | | |
| Sodium selenite pentahydrate | 0.03151% | CAS No.: | 26970-82-1 |
| Nickel(II) chloride hexahydrate | 0.00020% | | 7791-20-0 |
| Disodium molybdate | 0.00020% | | 7631-95-0 |
| Borax | 0.00020% | | 1330-43-4 |
| Zinc sulfate heptahydrate | 0.00012% | | 13986-24-8 |
| Cobalt(II) chloride hexahydrate | 0.00158% | | 7791-13-1 |
| Copper(I) chloride | 0.00016% | | 7758-89-6 |
| Potassium permanganate | 0.00028% | | 7722-64-7 |

In a second production variant, dolomite was used as expansion agent; the CO₂ eliminated during heating should also not alter the redox potential of the magnetite, but introduce calcium and magnesium in readily soluble form into the growth support.

Premixture One

| | |
|---|---|
| Iron oxide 316 | 14.4544% |
| Glass powder series | 64.7481% |

Premixture Two

| | | | |
|---|---|---|---|
| Dolomite | 1.6355% | | |
| Waterglass | 19.1274% | | |
| Water, hot | | | |
| Sodium selenite pentahydrate | 0.03168% | CAS No.: | 26970-82-1 |
| Nickel(II) chloride hexahydrate | 0.00020% | | 7791-20-0 |
| Disodium molybdate | 0.00020% | | 7631-95-0 |
| Borax | 0.00020% | | 1330-43-4 |
| Zinc sulfate heptahydrate | 0.00012% | | 13986-24-8 |
| Cobalt(II) chloride hexahydrate | 0.00158% | | 7791-13-1 |
| Copper(I) chloride | 0.00016% | | 7758-89-6 |
| Potassium permanganate | 0.00028% | | 7722-64-7 |

In both exemplary embodiments, the green grain produced from the starting mixture in a granulation process was dried in a rotary kiln at a temperature of 120° C. and this was followed by classification with a sieve limit of 0.25 mm.

The green bodies obtained were mixed with from 10 to 15% by mass of separating agent and foamed in a directly heated rotary kiln at temperatures between 780° C. and 815° C. with a passage time of from 10 to 15 minutes.

As separating agent, bentonite was also used in addition to kaolin. After foaming and cooling, the granular material was broken and sieved to a grain size of 0.25-1.5 mm.

### 3. EXPERIMENTAL STUDIES TO ASSESS THE GRANULAR EXPANDED-GLASS MATERIAL AS GROWTH SUPPORT IN BIOGAS PRODUCTION

### 3.1. Solubility of the Minerals and Trace Elements

For the trace elements and minerals fused into the growth support to become bioavailable for the microorganisms in the fermenter, it is necessary that they specifically go into solution in water or aqueous media under the conditions of this method.

To obtain the dissolution behavior over time, the test material was subjected to an elution.

In the elution test according to DEV S4, the samples, solid, pasty and sludgy materials, are slowly inverted or shaken for 24 hours in distilled water. The liquid/solid ratio is to be adjusted to a ratio of 10/1. In the course of this, the sample is to remain in constant motion, but further comminution, for example due to abrasion, is to be avoided.

In principle, it is assumed with this approach that the substances to be determined are soluble in water. To answer particular questions, it may be appropriate to use elution liquids other than distilled water, and so it is also possible to use solutions of defined pH as eluent.

In a first test run, what was attempted was to adjust the pH to 8 on a daily basis. The background to this test setup was that the pH values in an optimally running fermenter are within a range of 7-8.

Sample number

| | | 11-24616 | 11-24617 |
|---|---|---|---|
| | | V111115 | V111115 |
| | | eluate after | eluate after |
| Parameter | Standard | 1 day | 4 days |
| Eluate | DIN EN 71-3 | Done | Done |
| according to | | | |
| DIN EN 71-3 | | | |
| Silicon ICP- | DIN EN ISO | 9.9 mg/l | 9.9 mg/l |
| OES | 11885 E22 | | |
| Sodium | DIN EN ISO | 108 mg/l | 118 mg/l |
| | 11885 E22 | | |
| Calcium ICP- | DIN EN ISO | 39 mg/l | 49 mg/l |
| OES | 11885 E22 | | |
| Magnesium | DIN EN ISO | 16 mg/l | 33 mg/l |
| ICP-OES | 11885 E22 | | |
| Iron ICP-OES | DIN EN ISO | 0.39 mg/l | 0.41 mg/l |
| | 11885 E22 | | |
| Cobalt ICP- | DIN EN ISO | <0.01 mg/l | <0.01 mg/l |
| OES | 11885 E22 | | |
| Copper ICP- | DIN EN ISO | 0.039 mg/l | 0.070 mg/l |
| OES | 11885 E22 | | |
| Selenium ICP- | DIN EN ISO | 0.042 mg/l | 0.046 mg/l |
| OES | 11885 E22 | | |
| Manganese | DIN EN ISO | 0.026 mg/l | 0.041 mg/l |
| ICP-OES | 11885 E22 | | |
| pH | DIN 38404-C5 | 6.65 | 9.85 |

Table 3: Dissolved proportions after 1 day and 4 days in the elution test with pH adjustment around 8.

It was found that, in a slightly basic environment, dissolution of the trace elements starts very much more rapidly, and that the alkalis and alkaline earths were, however, slightly delayed in their solubility. This result largely confirms the theoretical principles of glass corrosion.

In the second eluate approach, the test preparation was adjusted once with acetic acid to a pH of 5, and both the dissolved substances and the ensuing pH were determined over a period of 45 days. With this test setup, the aim was to determine not only the solubility, but also additionally the buffering capacity of the growth supports in the fermenter.

If a fermenter starts to acidify owing to process disturbances (see under “1.1 Biogas principles” and “1.3. Growth rates of microorganisms”), the alkalis and alkaline earths going into solution from the growth support according to the invention counteract the acidification.

TABLE 4
Dissolved proportions after 1 day and 3 days in the elution
test with one-off pH adjustment to 5.

| | | Sample number | |
|---|---|---|---|
| | | 11-08862 | 11-08863 |
| | | Eluate from | Eluate from |
| Parameter | Standard | May 17, 2011 | May 19, 2011 |
| Eluate | DIN EN 71-3 | Done | Done |
| according to | | | |
| DIN EN 71-3 | | | |
| Silicon ICP- | DIN EN ISO | 7.12 mg/l | 7.2 mg/l |
| OES | 11885 E22 | | |
| Sodium | DIN EN ISO | 136 mg/l | 141 mg/l |
| | 11885 E22 | | |
| Calcium ICP- | DIN EN ISO | 17.3 mg/l | 25.6 mg/l |
| OES | 11885 E22 | | |
| Magnesium | DIN EN ISO | 25.7 mg/l | 25.3 mg/l |
| ICP-OES | 11885 E22 | | |
| Iron ICP-OES | DIN EN ISO | <0.01 mg/l | <0.01 mg/l |
| | 11885 E22 | | |
| Cobalt ICP- | DIN EN ISO | <0.01 mg/l | <0.01 mg/l |
| OES | 11885 E22 | | |
| Copper ICP- | DIN EN ISO | <0.01 mg/l | <0.01 mg/l |
| OES | 11885 E22 | | |
| Selenium ICP- | DIN EN ISO | 0.024 mg/l | 0.043 mg/l |
| OES | 11885 E22 | | |
| Manganese | DIN EN ISO | <0.01 mg/l | <0.01 mg/l |
| ICP-OES | 11885 E22 | | |
| pH | DIN 38404-C5 | 7.15 | 9.40 |

On the test fermenters, it was possible to detect that the methanogenic archaea in biofilms which had formed on the growth supports still survived even in a fermentation broth having a pH between 5 and 6. After appropriate measures to raise the pH to 8, biogas formation emanating from the growth supports restarted very rapidly.

### 3.2 Colonization Experiments for Different Growth Supports

Hohenheim Biogas Test

The so-called HBT test was developed by the University of Hohenheim; it serves for the evaluation of different substrates and input materials and for the representation of different method-related basic conditions in fermenters of biogas plants or wastewater treatment plants. Since up to 200 samples can be used in the sampler carrier, an adequate statistical coverage is possible by means of this test.

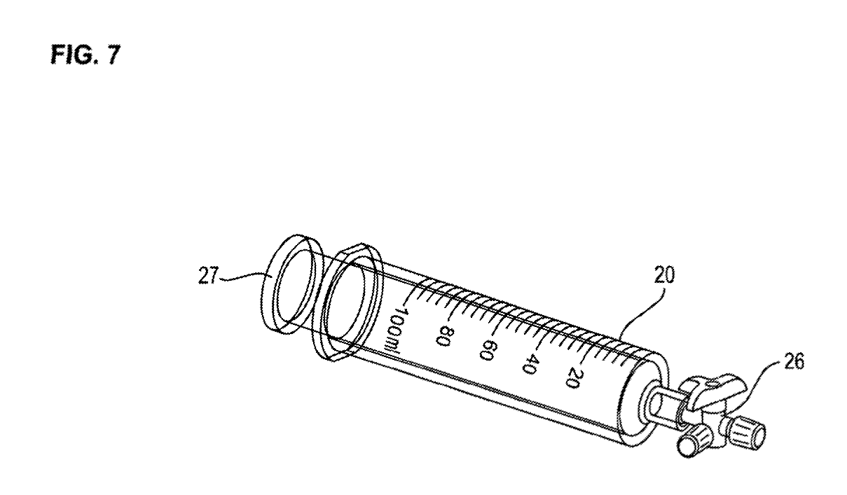

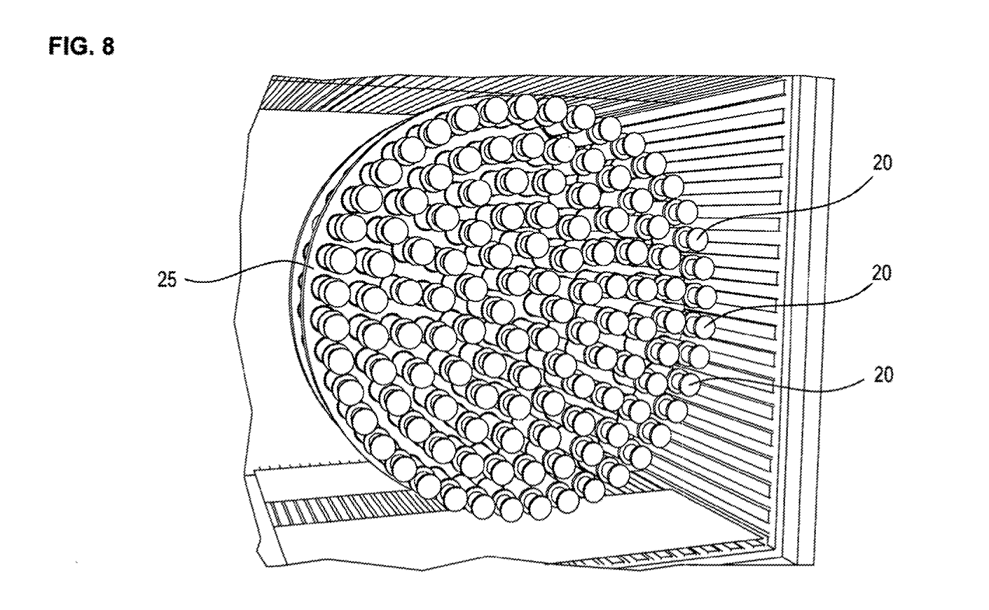

The core elements of the test setup are reaction vessels in the form of syringes 20 made of glass (FIG. 7) and a device for storing said syringes 20, the rotary frame 25 (FIG. 8). The syringes 20 have a fill volume of 100 ml and are provided with a three-way valve 26. In the rotary frame 25, the syringes can be stored horizontally. It is provided with an adjustable drive which allows rotation of the rotary frame 25 around the longitudinal axis of the syringes stored therein. The rotation can be carried out with speeds in the range from 5 to 50 min⁻¹. The rotary frame 25 and a fermenter containing an inoculation culture are accommodated in a heatable climatic chamber.

Filled into the syringes 20 were test preparations consisting of the inoculum (monosubstrate from sugar beet silage from the preliminary experiments) and, in each case, one variety of the different growth supports. In addition, control preparations without growth supports were also tested. The volume of the preparations in the syringes and the mass concentration of the growth supports in the preparation were kept constant within a test series. The volume of the preparations must be distinctly below the maximum fill volume of the syringe 20, since residual volume is additionally required for the biogas formed by the preparation. To allow the ambient air carried in during the first filling to escape, the three-way valve 26 is opened and the plunger 27 of the syringe 20 is pushed in up to the volume occupied by the preparation. The syringes 20 filled with the preparations were stored in the rotary frame 25 over the entire duration of a test series. For the maintenance of the process temperature, the climatic chamber was adjusted in temperature to 37° C.

Shown below are the study results of the colonization experiments for growth supports with fused trace elements in comparison with selected growth supports from the preliminary experiments. The following sample designations are used throughout:

a) B1: Granular material with magnetite, without calcium, corresponds to type 7 from the preliminary experiments;
b) B2: like B1, but with dolomite as expansion agent, corresponds to type 8 from the preliminary experiments;
c) B3: like B1 with addition of trace elements (according to first production variant);
d) B4: like B1 with addition of trace elements with dolomite as expansion agent (according to second production variant).

Microbiological Studies—Molecular Genetics Analysis

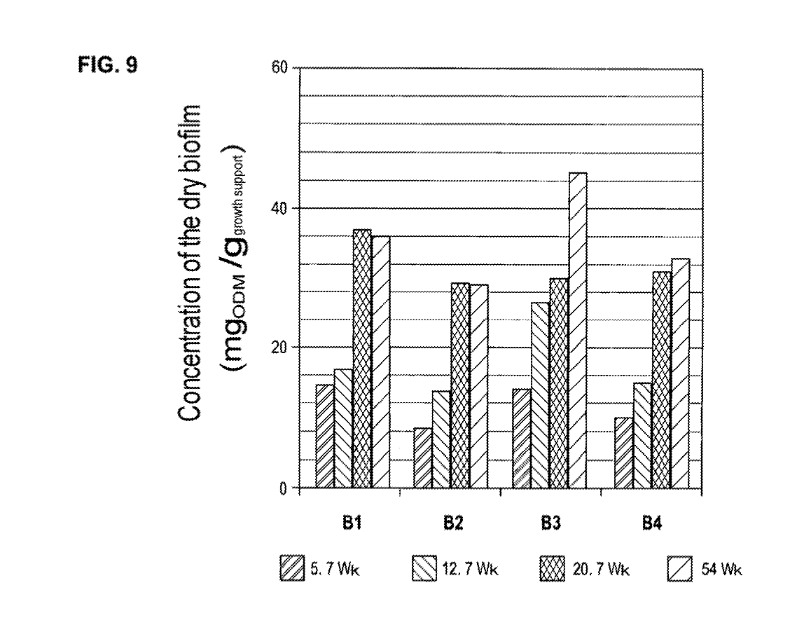

FIG. 9 to FIG. 11 depict the development over time of the biofilm on the growth supports within a test period of 54 weeks, with the syringes 20 being sampled after 5.7 weeks (“5.7 wk”), 12.7 weeks (“12.7 wk”), 20.7 weeks (“20.7 wk”) and 5 weeks (“54 wk”).

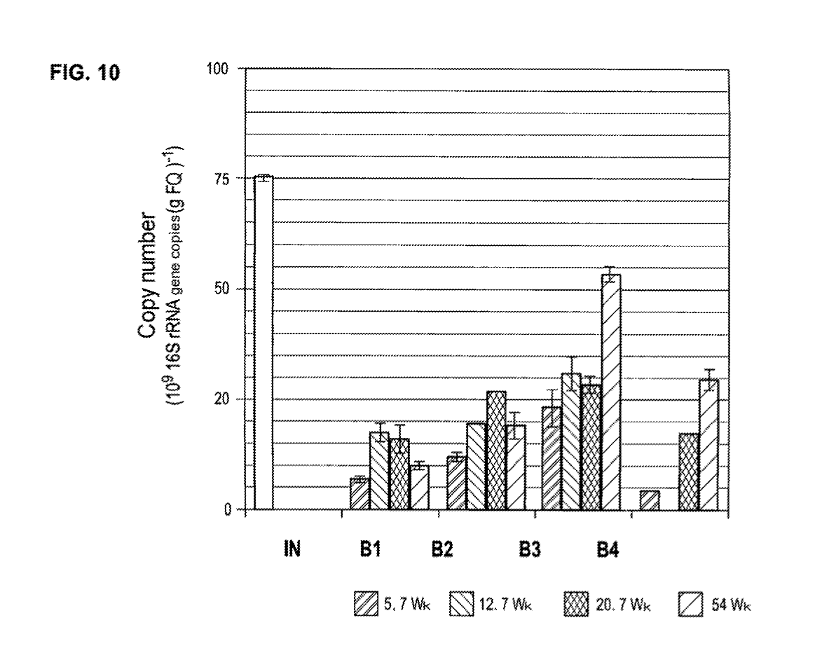

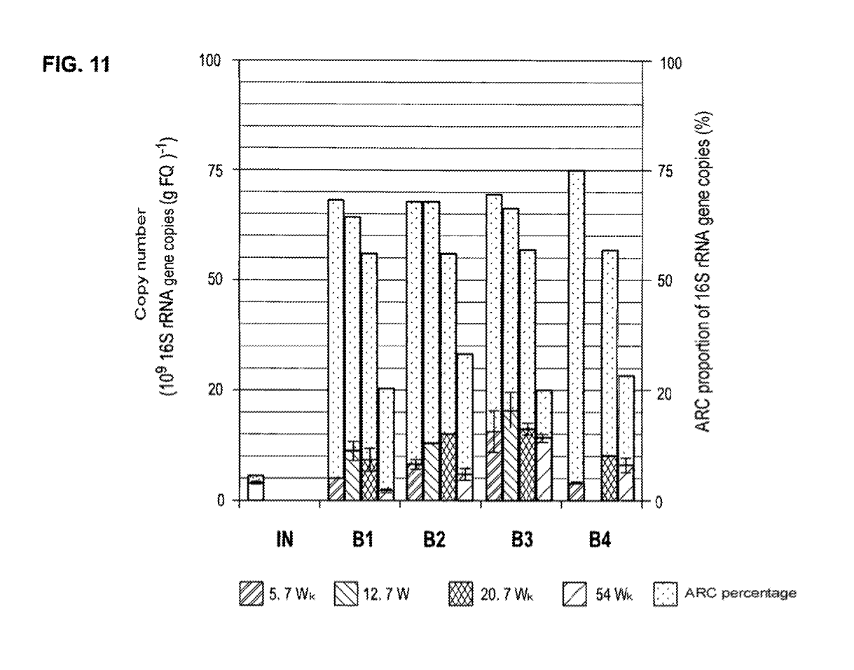

The values for concentration of the dry biofilm on the growth supports in (mg_{ODM}/g_{growth support}) are plotted in FIG. 9, the total microbial population (BAC and ARC) in (10⁹ 16 S rRNA gene copies (gFQ)⁻¹) are plotted in FIG. 10, and the occurrence of archaea (ARC) in (10⁹ 16 S rRNA gene copies (gFQ)⁻¹) (left-hand scale) and the proportion thereof of the total population in % (right-hand scale) are plotted in FIG. 11. The values for the inoculum (IN) are likewise depicted in FIG. 10 and FIG. 11.

It is apparent when viewing the charts that the biofilm mass was almost exclusively determined by the two factors “Growth support material type” and “Time”.

It was noticeable that the total population in the case of the specimens B1 and B2 rose up to about the 20th week and then decreased. In the case of the specimens B3 and B4, it was possible to observe a constant rise to distinctly higher values over the entire observation period. The 12-weeks value for B4 had to be discarded because of an error during the sampling. The proportion of methanogenic organisms follows substantially the same pattern. However, the archaea percentage of the total population distinctly fell from the 20th week in all the specimens studied.

This discovery cannot be conclusively explained at present. However, it is suspected therefrom that, firstly, the use of growth supports with trace elements promotes the colonization and multiplication of microorganisms and, secondly, the low reproduction capacity of the methanogens (see “1.3. Growth rates of microorganisms”) also appears to play a role here. The diversity of the various growth support types with respect to methanogenic organisms proved to be homogeneous.

### 3.3 Biogas Production

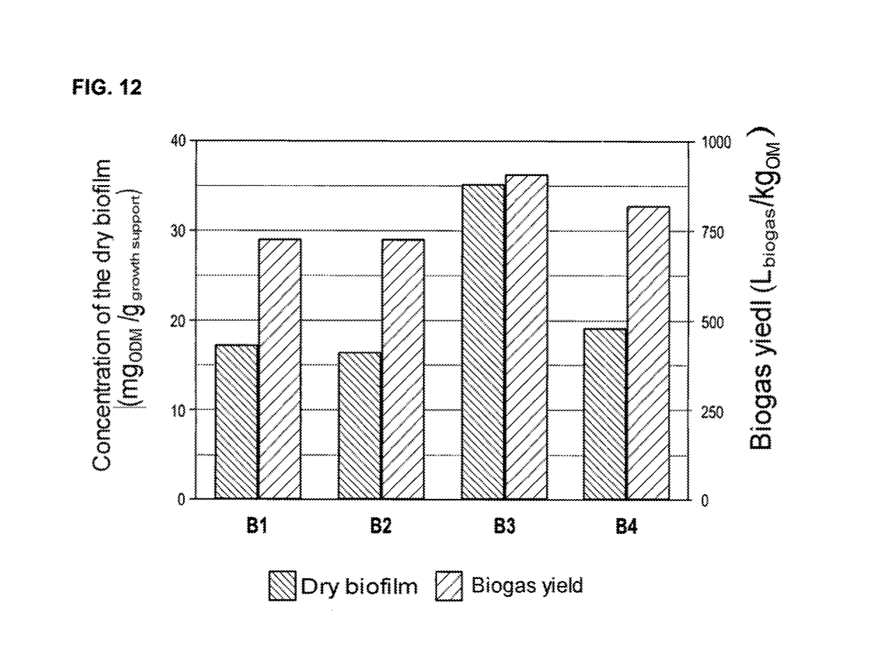

In order as well to be able to provide information about biogas formation from the individual material types, the concentration of the dry biofilm on the growth supports was determined after a growth time of 33 days (mg_{ODM}/_{ggrowth support})—FIG. 12, left-hand scale, white bars—and compared with the respectively achieved biogas yield (L_{biogas}/kg_{OM})—FIG. 12, right-hand scale, gray bars.

From FIG. 12, it is possible to deduce a connection between the biogas yield and the concentration of the dry biofilm on the different growth supports.

The growth supports B3 and B4, in which trace elements are fused, appear, as already established above, to promote biofilm formation and to achieve a higher biogas yield as a result. The lower biofilm mass in the case of the sample B4 with simultaneously high biogas yield indicates, according to this first evaluation, that the microorganisms present in the sample had a relatively high metabolic activity at the time of measurement. This behavior for biofilms is known. When a population which has merged to form a colony in a biofilm is provided with sufficient nutrition and optimal life conditions, the microorganisms respond with an enhanced metabolic activity or the biofilms redissolve in some cases and migrate into the fermentation liquid.

### 3.4 Confirmation Experiments

In order to able to assess the influence and the interactions between growth support and the suspended microbial biomass, a further experiment was prepared in which both the different growth supports and the fermentation liquid thereof was evaluated after two and twelve weeks. (Results not shown)

For almost all the growth supports, the number of gene copies was in the same order of magnitude of the prior experiment. From a molecular genetics perspective, they can thus be considered to be approximately the same. The arachaea proportion of the total population distinctly increased for all the growth supports. In the case of growth support specimen B3, the strongest increase with incubation time occurred—especially in the archaea population.

In the case of B1, a strong decrease in the microorganism population was established. This could indicate a detachment of the biofilm from the surface of the growth supports, meaning that saturation had already occurred and the maximum ratio between growth support and biofilm is between 40 and 50 mg of ODM per g of growth support.

In the fermentation liquid, it was possible to detect similarly high total populations of microorganisms as on the growth supports. In the case of the preparations B2 to B4, the archaea population and the proportion thereof of the total population increased with incubation time, though the values for the growth supports were not reached. It was not possible to identify a strong promotion of methanogenic organisms in the fermentation liquid through the use of the growth supports.

### 3.5. Microscopic Studies

The microscopic studies (not shown) yielded important conclusions which can be used to assess the suitability of the different growth supports. The degree and the form of the colonization of the growth supports could be easily assessed on the basis of DNA staining (fluorescence microscopy) of the microorganisms present. The occurrence of methanogenic organisms could be assessed on the basis of their autofluorescence. For each of the growth supports studied, autofluorescence also occurred in all the microorganism colonies detected on the basis of DNA staining. Thus, methanogenic organisms were represented in all the microorganism colonies of the biofilms.

Experiments with growth support type 1 (the preliminary experiments) and B3 (with fused trace elements) after multi-month use in test fermenters which were fed with beet silage were compared. Whereas very many growth supports are colonized only very poorly to not at all in the case of type 1, the specimen B3 shows a virtually extensive colonization. It can be clearly seen that methanogenic microorganisms also preferentially settle on the growth supports with trace elements. The strong intensity for the UV autofluorescence is an indication of a high metabolic activity of this population.

### 4. CONCLUSION

In summary, it can be stated for the growth supports according to the invention:

a) Growth supports are fundamentally suitable as support for biogas-forming and methane-forming biofilms.
b) The particle-free control fermenters were the least stable variant in the entire course of experiments. Thus, the positive effect of growth supports on the stability of the biogas process can be considered to be verified.
c) The growth supports must be broken prior to use in a fermenter/bioreactor and sieved to a defined grain-size distribution. The resulting irregular surface structure accommodates very well the formation of biofilm.
d) The development of the adherent biofilm mass is considerably determined by the nature of the growth support and by the residence time in the fermenter.
e) The mass of the biofilm adhering to a growth support does not, on its own, provide information about the quality of the biofilm. Molecular genetics analysis allows a deeper insight.
f) The settlement of microorganisms on the surfaces of the growth supports is highly selective—the proportion of methanogenic organisms is distinctly higher than in the surrounding fermentation liquid or the inoculum.
g) The fusion of trace elements and minerals into the growth supports has, depending on the substrate, a positive effect in the case of sufficient incubation time.
h) Under the basic conditions in a biogas fermenter/bioreactor, the trace elements and minerals pass into solution over a long period. In the case of the entertained quantity of approximately 2% growth support of the total filling weight of a fermenter/bioreactor, an overloading or even process inhibition due to “critical” trace elements appears to be ruled out.
i) The growth supports introduce a considerable buffering capacity into the system and thus counteract any process disturbances due to acidification.
j) A “microenvironment” which is clearly optimal for the biofilms forms on the surface of the growth supports, which microenviroment allows the archaea to survive even in an acidified environment.

The invention becomes particularly clear from the above-described exemplary embodiments, but is nevertheless not restricted thereto. On the contrary, further embodiments of the invention can be derived from the claims and the above description.

### List of Abbreviations/Glossary

Acetogenesis: formation of acetic acid

Acidogenesis: formation of (organic) acids

Acidophile: acid-loving

Acetoclastic: acetic acid-degrading

Anion: negatively charged atom or molecule

ATP: adenosine triphosphate (universal biochemical energy carrier)

Biocenosis: (from the Ancient Greek βioç bios “life” and κoivóç koinós “together”) is a community of organisms of different species in a delimitable living space, in this case in the fermenter, bioreactor.

BtL Biomass to Liquid

Cellulolytic: cellulose fiber-dissolving/degrading

COD: chemical oxygen demand, measure of the sum of all substances present (in water) and oxidizable under certain conditions

DGGE: denaturing gradient gel electrophoresis, method for the specific detection of certain DNA segments with the aid of the running behavior in a gel with a gradient of denaturing substance

Disproportionation: can be observed particularly frequently in redox reactions. It is then a reaction in which the same compound is oxidized and reduced at the same time and the oxidation states of multiple atoms of the same type are changed in different directions. Disproportionation of chlorine (oxidation state of the chlorine atoms in Cl₂=zero) in sodium hydroxide solution to give chloride (−1) and hypochlorite (+1)

Diversity: number of different taxa (usually species) in a population

DNA: deoxyribonucleic acid, carrier of hereditary material

e−: electron(s)

EDTA: ethylenediaminetetraacetic acid

Electron acceptor: element which takes up electrons through change in its valency

Electron donator: element which releases electrons through change in its valency

Electron carrier: molecule which transfers electrons for the reduction of another molecule

Ergodicity: refers to the average behavior of a system. Such a system is described by a model function which defines the temporal development of the system depending on its current state

VOA/TIC: ratio of volatile organic acids to inorganic carbonate, =parameter for estimating the stability of the biocenosis; acidification is indicated from about 0.6

Green body: ceramics term for an unfired/unsintered blank.

Halophile: salty conditions-loving

HBT: Hohenheim biogas test

Hydrogenotrophic: hydrogen-utilizing for growth

Hydrolysis: cleavage of molecules under reaction with water

Hydrolytic stability: resistance of glass surface to aqueous attack

Immobilization: the spatial fixation of bacteria, cells or enzymes in gel particles, capsules or else in defined reaction spaces. Immobilization leads to a transfer of the catalytic activity of submicroscopically and microscopically small units to macroscopically identifiable particles in order to achieve retention

Inoculum: this term is understood in biotechnology to mean the quantity of cells used to inoculate a fermenter

Cosubstrate: waste and feed remains, but also specific energy plants for biogas preparation

Cation: positively charged atom or molecule

Lignocellulose: compound composed of cellulose with (incorporated) lignin

LFL: Bayerische Landesanstalt für Landwirtschaft [Bavarian State Research Center for Agriculture]

mcrA/mrtA: gene for subunit A of the key enzyme for methane formation

Mesophilic: average temperature level-loving (approximately 30-40° C.

Methanogenesis: formation of methane

Methanogenic archaea: methane gas-forming microorganisms

Module: the ratio of silicon oxide and alkali oxide in waterglass

Network formers: also called glass formers, form the molecular basic structure of glass. For glass formation, no further substances are required. For example, quartz glass has SiO₂ as the sole constituent

Network modifiers: compounds which form a glass together with one or more network formers. Network modifiers alter the structure and properties of the glass

OLR: organic loading rate

ODM: organic dry matter

Oxidation: change in state of a molecule with electron release

PCR: polymerase chain reaction, molecular biology method for amplifying certain DNA segments for their specific detection

Polar molecule: molecule having a positively and negatively charged end

Proteases: protein-cleaving enzymes

Loading rate: is the quantity of organic dry matter (ODM) in kilograms per m³ of fermenter volume per day

Reduction: change in state of a molecule with electron upake

Sequence: sequence of bases (adenine, cytosine, guanine, thymine) in the DNA

Sequencing: determination of the sequence of bases in the DANN examined (using different methods)

Species: taxonomical term, close relationship: >97.5%

TE: trace element cocktail with defined composition

SSCP: single strand conformation polymorphism, method for the specific detection of certain DNA segments with the aid of the folding of single-stranded DNA

Stabilisers: can be both network modifier and network former in glass. However, they are not capable of forming a glass as single component

Substrate: material which is fermented in a biogas plant

Supplementation: addition

Syntrophic: jointly growing, dependent on each other in nutrient utilization

Taxon: (singular; plural: taxa; from the Greek Tabic taxis (order, rank) refers in biology to a group of organisms that is identifed as a systematic unit

Thermophilic: high temperature level-loving (approximately 45-65° C.)

### LIST OF REFERENCES

Agraferm technologies, Patent No. DE102007061138A1 Jun. 25, 2009.
Bauer Ch, Lebuhn M, Gronauer A—Mikrobiologische Prozesse in landwirtschaftlichen Biogasanlagen [Microbiological processes in agricultural biogas plants]. Schriftenreihe der Bayerischen Landesanstalt für Landwirtschaft [Publication series of the Bavarian State Research Center for Agriculture]. September 2009, Freising-Weihenstephan.
Bertram, P. A.; Schmitz, R. A.; Linder, D.; Thauer, R. K.: Tungstate can substitute for molybdate in sustaining growth of Methanobacterium thermoautotrophicum. In: Microbiol. 161, 1994: pp. 220-228.
Bischofsberger W., Dichtl N., Rosenwinkel K.-H., Seyfried C. F., Böhnke B.: Anaerobtechnik [Anaerobe technology], Springer Verlag Berlin, Heidelberg 2005, 2nd edition.
Chasteen, T. G.; Bentley, R.: Biomethylation of selenium and tellurium: In: Microorganisms and plants. Chem. Rev. 103, 2003: pp. 1-25.
Deppenmeier U., Lienard T., Gottschalk G. (1999). Novel reactions involved in energy conservation by methanogenic archaea. FEBS Lett. 457, 291-297.
Effenberger M., Lebuhn M., Gronauer A. (2007). Fermentermanagement—Stabiler Prozess bei NawaRo-Anlagen [Fermenter management—stable process in reneweable resource plants]. Kongressband der 16. Jahrestagung des Fachverbands Biogas e. V.: Biogas im Wandel [Conference transcript of the 16th annual meeting of the professional association Biogas e.V.: biogas changes], Jan. 31, 2007 to Feburary 2, 2007, Leipzig, 99-105.
Entrup N. L., Oehmichen J.: Lehrbuch des Pflanzenbaus [Textbook of plant cultivation], Th. Mann Verlag, Bonn 2008.
Gülzower Fachgesprache, Band 35 [Gülzower technical discussions, volume 35]—Einsatz von Hilfsmitteln zur Steigerung der Effizienz und Stabilität des Biogasprozesses [Use of auxiliares to increase the efficiency and stability of the biogas process]. September 2010.
Haun E., Reihenuntersuchungen im Batchverfahren zum Einfluss von Substratqualitat und Spurenelementen auf den anaeroben biologischen Abbau [Serial studies in the batch method relating to the influence of substrate quality and trace elements on anaerobic biodegradation]. Abschlussbericht ISAH, Leibniz Universitat Hannover [Final report from the ISAH, Leibniz University of Hanover]. 04.2008.
Hölker U., Das Salz in der Suppe [The salt in the soup]. Special print from Joule, 5.2010.
Kienberger Th., Karl J., Institut für Wärmetechnik [Institute of Thermal Engineering], TU Graz [Graz University of Technology], 2010, www.iwt.tugraz.at.
Klocke, M.; Mündt, K.; Sontag, M.; SchOnberg, M.; Linke, B.: Mikrobielle Lebensgemeinschaften in einem zweistufigen Biogasreaktor mit Bioleaching und Roggensilage [Microbial communities in a two-step biogas reactor with bioleaching and rye silage]. Wie viel Biogas steckt in Pflanzen? Abschluss-Symposium des Biogas Crops Network (BCN) [How much biogas is there in plants? Closing symposium of the Biogas Crops Network (BCN)]. Potsdam Bornim: Leibnitz-Institut für Agrartechnik Potsdam Bornim e. V. [Potsdam Bornim: Leibnitz Institute for Agricultural Engineering Potsdam Bornim e.V.], Bomimer Agrartechnische Berichte [Bornim agricultural engineering reports], issue 68, 2009, pp. 126-139.
Krätzer Ch., Substratumsetzung und Schutz vor Sauerstoffradikalen in Methanosarcina mazei [Substrate conversion and protection from free oxygen radicals in Methanosarcina mazei].
Dissertation from Friedrich-Wilhelms-Universität Bonn [University of Bonn] 2011.
Lebuhn M., Liu F., Heuwinkel H., Gronauer A. (2008b). Biogas production from monodigestion of maize silage—long-term process stability and requirements. Water Sci. Tech. 58(8), 1645-1651.
Lebuhn M., Gronauer A. (2009). Microorganisms in the biogas-process—the unknown beings. Agric. Engin. (Landtechnik) 64/2, 127-130.
Lindorfer, H.: Verbesserung der Wirtschaftlichkeit von Biogasanlagen durch die Optimierung der Fermenterbiologie [Improving the economic viability of biogas plants through optimization of fermentation biology], manuscript—biogas sypmosium of the biogas working group at TBV e.V. on May 28, 2009-.
SRU (editor)—Sachverständigenrat für Umweltfragen: Klimaschutz durch Biomasse—Sondergutachten [German Advisory Council on the Environment: Climate protection via biomass—special report] July 2007. Berlin: Erich Schmidt Verlag GmbH & Co., 2007.
Ternes W., Täufel A., Tunger L, Zobel, M.: Lebensmittellexikon [Food dictionary], Behr's Verlag Hamburg, Munich 2005.
Weiland, P: Biogas ein zukunftsweisender Energieträger [Biogas: a forward-looking energy source]. Erneuerbare Energie in der Land(wirt)schaft 2001 [Renewable energy in the countryside/agriculture 2001]. Zeven: Verlag für landwirtschaftliche Publikationen [Zeven: publisher of agricultural publications], M. C. Medenbach, 2001.
Dr. rer. nat. Merrettig-Bruns Ute, Fraunhofer-Institut für Umwelt-, Sicherheits-and Energietechnik [Fraunhofer Institute for Environmental, Safety, and Energy Technology]: Test zur Messung der Stoffwechselaktivität der Bakterien im Gärbehalter [Test for measuring bacterial metabolic activity in fermentation vessels].

The following is a summary list of reference numerals and the corresponding structure used in the above description of the invention:
1 Biomass
2 Primary fermentation
3 Hydrolysis
4 Monomer
5 Building block
6 Acidogenesis
7 Acetogenesis
8 Secondary fermentation
9 Acetate
10 Methanogenesis
11 Anaerobic respiration
12 Biogas
20 Syringe
25 Rotary frame
26 Three-way valve
27 Plunger

1. A process for producing a granular expanded-glass material, which comprises the following process steps of:
mixing a glass powder, an expansion agent and a binder to form a starting mixture;
adding minerals and/or trace elements to the starting mixture which supply nutrients for microorganisms;
granulating the starting mixture to form granular expanded-glass material green bodies; and
foaming the granular expanded-glass material green bodies to form granular expanded-glass material particles at temperatures of from 600° C. to 950° C.

2. The process according to claim 1, which further comprises selecting the minerals added from the group consisting of carbon, nitrogen, phosphorus, sulfur, sodium, calcium, magnesium and iron.

3. The process according to claim 1, which further comprises selecting the trace elements from the group consisting of elements based on cobalt, manganese, molybdenum, nickel, selenium, tungsten and zinc.

4. The process according to claim 1, which further comprises providing a ground soda-lime glass as the glass powder.

5. The process according to claim 1, which further comprises providing an alkali silicate waterglass as the binder.

6. The process according to claim 1, which further comprises adding a separating agent in a form of calcium carbonate, dolomite and/or bentonite to the granular expanded-glass material green bodies prior to foaming for an additional supply of additional minerals.

7. The process according to claim 1, which further comprises using at least one of the following as the minerals and the trace elements with corresponding proportions by weight:

| | |
|---|---|
| Sodium selenite pentahydrate | 0.02000-0.04000%; |
| Nickel(II) chloride hexahydrate | 0.00010-0.00030%; |
| Disodium molybdate | 0.00010-0.00030%; |
| Borax | 0.00010-0.00030%; |
| Zinc sulfate heptahydrate | 0.00010-0.00020%; |
| Cobalt(II) chloride hexahydrate | 0.00100-0.00200%; |
| Copper(I) chloride | 0.00010-0.00030%; and |
| Potassium permanganate | 0.00020-0.00040%. |

8. The process according to claim 4, wherein the ground soda-lime glass has a proportion by weight of from 60% to 70%.

9. The process according to claim 5, wherein the alkali silicate waterglass has a proportion by weight of from 15% to 25%.

10. The process according to claim 6, which further comprises selecting the additional minerals from the group consisting of calcium, magnesium and iron.

11. A granular expanded-glass material for use in a biogas plant or an anaerobic wastewater treatment plant, comprising:
a glass powder;
an expansion agent;
a binder; and
an addition of minerals and/or trace elements.

12. The granular expanded-glass material according to claim 11, wherein said minerals present are carbon, nitrogen, phosphorus, sulfur, sodium, calcium, magnesium and/or iron.

13. The granular expanded-glass material according to claim 11, wherein said trace elements are based on cobalt, manganese, molybdenum, nickel, selenium, tungsten and/or zinc being present.

14. The granular expanded-glass material according to claim 11, further comprising a separating agent in a form of calcium carbonate, dolomite and/or bentonite.

15. The granular expanded-glass material according to claim 11, wherein at least one of the following are selected as said minerals and said trace elements with corresponding proportions by weight:

| | |
|---|---|
| Sodium selenite pentahydrate | 0.02000-0.04000%; |
| Nickel(II) chloride hexahydrate | 0.00010-0.00030%; |
| Disodium molybdate | 0.00010-0.00030%; |
| Borax | 0.00010-0.00030%; |
| Zinc sulfate heptahydrate | 0.00010-0.00020%; |
| Cobalt(II) chloride hexahydrate | 0.00100-0.00200%; |
| Copper(I) chloride | 0.00010-0.00030%; and |
| Potassium permanganate | 0.00020-0.00040%. |

16. The granular expanded-glass material according to claim 11, wherein:
said glass powder is a soda-lime glass powder; and
said binder is alkali silicate waterglass.

17. A method of operating a bioreactor, which comprises the steps of:
providing a granular expanded-glass material containing a glass powder, an expansion agent, a binder, and an addition of minerals and/or trace elements; and
using the granular expanded-glass material as a growth support for microorganisms in the bioreactor.

18. The method according to claim 18, wherein the bioreactor is a biogas plant or an anaerobic wastewater treatment plant.
